(19) United States
(12) Patent Application Publication
(10) Pub. No.: US 2024/0141390 A1
(43) Pub. Date: May 2, 2024

(54) METHOD FOR PREPARING FIBER MATERIAL FROM BANANA PSEUDOSTEM

(71) Applicant: Fu-Hsiang YANG, Yuanlin City (TW)

(72) Inventor: Fu-Hsiang YANG, Yuanlin City (TW)

(21) Appl. No.: 18/191,201

(22) Filed: Mar. 28, 2023

(30) Foreign Application Priority Data
Oct. 28, 2022 (TW) 111141185

(51) Int. Cl. C12P 1/02; D01C 1/00

(52) U.S. Cl. CPC C12P 1/02; D01C 1/00; C12R 2001/865

(57) ABSTRACT

A method for preparing a fiber material from a banana pseudostem, which includes subjecting the banana pseudostem to a pressing treatment to obtain a pressed banana pseudostem, subjecting the pressed banana pseudostem to a fermentation reaction with Saccharomyces cerevisiae to obtain a fermented culture, subjecting the fermented culture to a simultaneous saccharification and fermentation process with pectinase to obtain a fermented product, subjecting the fermented product to a solid-liquid separation treatment to obtain a solid fraction, and subjecting the solid fraction to an alkali treatment, a water washing treatment, a bleaching treatment, a softening treatment, and a drying treatment in sequence, so as to obtain the fiber material.

### CROSS-REFERENCE TO RELATED APPLICATION

This application claims priority of Taiwanese Invention Patent Application No. 111141185, filed on Oct. 28, 2022.

### FIELD

The present disclosure relates to a method for preparing a fiber material from a banana pseudostem.

### BACKGROUND

The banana plant (scientific name: Musa×paradisiaca) is a herbaceous perennial of the family Musaceae, and the fruit of the banana plant is the fourth most important fruit crop in the world. After the banana fruit is harvested, the remaining banana pseudostem, which is discarded, will cause pest (such as ants and weevils) infestation, and the emission of greenhouse gases (such as methane) will occur during the decay of banana pseudostem. Therefore, those skilled in the art still strive to develop an effective way to reuse banana pseudostem waste, thereby reducing pest infestation and environmental pollution.

The cellulose, hemicellulose, and lignin contained in the banana pseudostem are intertwined and wrapped to form a complex and tough network structure. Therefore, the banana pseudostem must be subjected to a process for delignification before recycling. Methods commonly used for delignification include physical methods (such as scraping methods) and chemical methods (such as acid treatments and alkali treatments). However, these methods might cause problems such as poor fiber extraction, loss of fine fibers (such as cotton-like fibers), and low yields.

Recently, microorganisms and enzymes have been applied in the reprocessing of agricultural waste and the reuse of fibers. For instance, CN 111996603 A discloses a biological extraction method of pineapple leaf fiber, which includes subjecting a pineapple leaf fiber raw material to an enzyme treatment, an enzyme deactivation and rinsing treatment, an ultrasonic treatment, and a drying treatment in sequence. The enzyme used in the enzyme treatment may be selected from the group consisting of laccase, xylanase, and pectinase. Before the enzyme treatment, the pineapple leaf fiber raw material can be subjected to a microbial treatment using Aspergillus niger spore. In the examples of CN 111996603 A, the pineapple leaf fiber raw material was treated with various enzyme solutions, and then the resultant pineapple leaf fibers were analyzed for residual sericin, breaking strength, thickness, softness, and color. The results show that, in comparison with the use of pectinase alone, high quality pineapple leaf fiber can be obtained by using a combination of laccase and xylanase to treat the pineapple leaf fiber raw material, indicating that not all enzymes with delignification effect are suitable for extraction of pineapple leaf fiber.

In spite of the aforesaid, there is still a need to develop a method for preparing a fiber material from a banana pseudostem, which can effectively reduce banana pseudostem waste and increase fiber yield.

### SUMMARY

Therefore, in a first aspect, the present disclosure provides a method for preparing a fiber material from a banana pseudostem, which can alleviate at least one of the drawbacks of the prior art. The method includes:
(a) subjecting the banana pseudostem to a pressing treatment, so as to obtain a pressed banana pseudostem;
(b) subjecting the pressed banana pseudostem to a fermentation reaction with Saccharomyces cerevisiae, so as to obtain a fermented culture;
(c) subjecting the fermented culture to a simultaneous saccharification and fermentation process with pectinase, so as to obtain a fermented product;
(d) subjecting the fermented product to a solid-liquid separation treatment, so as to obtain a solid fraction; and
(e) subjecting the solid fraction to an alkali treatment, a water washing treatment, a bleaching treatment, a softening treatment, and a drying treatment in sequence, so as to obtain the fiber material.

In a second aspect, the present disclosure provides a fiber material which is prepared by a method as described above. Such fiber material can alleviate at least one of the drawbacks of the prior art.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a photograph showing the segmented banana pseudostems prepared in Example 1, infra.

FIG. 2 is a photograph showing the pressed banana pseudostems prepared in Example 1, infra.

FIG. 3 is a photograph showing the fermented product prepared in Example 1, infra.

FIG. 4 is a photograph showing the fiber material prepared in Example 1, infra.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for preparing a fiber material from a banana pseudostem, which includes:
(a) subjecting the banana pseudostem to a pressing treatment, so as to obtain a pressed banana pseudostem;
(b) subjecting the pressed banana pseudostem to a fermentation reaction with Saccharomyces cerevisiae, so as to obtain a fermented culture;
(c) subjecting the fermented culture to a simultaneous saccharification and fermentation process with pectinase, so as to obtain a fermented product;
(d) subjecting the fermented product to a solid-liquid separation treatment, so as to obtain a solid fraction; and
(e) subjecting the solid fraction to an alkali treatment, a water washing treatment, a bleaching treatment, a softening treatment, and a drying treatment in sequence, so as to obtain the fiber material.

In certain embodiments, the banana pseudostem may be obtained from a banana plant selected from the group consisting of Cavendish spp., Musa spp., Ensete spp., and combinations thereof. In an exemplary embodiment, the banana pseudostem is obtained from a banana plant of Cavendish spp.

As used herein, the terms “pseudostem” and “stubble” can be used interchangeably.

According to the present disclosure, the pressing treatment, the fermentation reaction, the simultaneous saccharification and fermentation process, the solid-liquid separation treatment, the alkali treatment, the water washing treatment, the bleaching treatment, the softening treatment, and the drying treatment may be performed using techniques well-known to those skilled in the art. It should be noted that the procedures and operating conditions for the aforesaid treatments may be adjusted according to practical requirements and factors (such as the water content and amount of the banana pseudostem).

In certain embodiments, in step (a), the pressing treatment is conducted using an oil pressure type presser, so that approximately 30% to 50% of the juice of the banana pseudostem can be removed.

In certain embodiments, before performing step (a), the banana pseudostem may be subjected to a segmentation treatment. In an exemplary embodiment, the banana pseudostem is segmented to have a length ranging from 60 cm to 100 cm.

As used herein, the term “fermentation” can be used interchangeably with the terms “culturing” and “cultivation”.

It should be noted that the procedures and operating conditions for the fermentation reaction may be adjusted according to practical requirements and factors (such as the content ratio of Saccharomyces cerevisiae to the pressed banana pseudostem), so as to achieve an optimum fermentation effect.

In certain embodiments, in step (b), the fermentation reaction may be conducted at a temperature ranging from 20° C. to 32° C. In an exemplary embodiment, the fermentation reaction is conducted at 28° C.

In certain embodiments, in step (b), during the fermentation reaction, a sugar may be added to adjust the sugar content. In an exemplary embodiment, the sugar content is maintained at 18 degrees Brix to 20 degrees Brix.

In certain embodiments, the sugar may be selected from the group consisting of glucose, fructose, sucrose, table sugar mainly composed of sucrose, and combinations thereof.

Examples of the table sugar mainly composed of sucrose may include, but are not limited to, rock sugar, white sugar, soft white sugar, and brown sugar. In certain embodiments, the table sugar mainly composed of sucrose is white sugar.

In certain embodiments, the fermented culture obtained in step (b) may be further subjected to a distillation treatment, so as to obtain bioethanol and a distillation residue which can be used as an organic fertilizer. In an exemplary embodiment, the distillation treatment is conducted at a temperature ranging from 78° C. to 100° C.

In certain embodiments, in step (c), the simultaneous saccharification and fermentation process may be conducted at a temperature ranging from 15° C. to 55° C. In an exemplary embodiment, the simultaneous saccharification and fermentation process is conducted at 50° C.

In certain embodiments, in step (c), the pectinase suitable for use in this disclosure may be obtained as commercial products. In an exemplary embodiment, the pectinase is a pectic enzyme (Manufacturer: North Mountain Supply) purchased from Bake n Brew Homebrew Supply.

In certain embodiments, in step (e), the alkali treatment may be conducted using an alkaline solution at a temperature ranging from 60° C. to 90° C. for 3 hours.

In certain embodiments, the alkaline solution may be selected from the group consisting of a potassium hydroxide solution, a sodium hydroxide solution, a calcium carbonate solution, and combinations thereof. In an exemplary embodiment, the alkaline solution is a potassium hydroxide solution having a concentration ranging from 2 wt % to 4 wt %. In another exemplary embodiment, the alkaline solution is a 2 wt % potassium hydroxide solution.

In certain embodiments, in step (e), the water washing treatment may be conducted by washing the solid fraction with a high-pressure water column having a pressure of 100 bar, so as to remove lignin.

In certain embodiments, in step (e), the bleaching treatment may be conducted using a bleaching agent at a temperature ranging from 20° C. to 150° C. for 3 hours.

In certain embodiments, the bleaching agent may be selected from the group consisting of a hydrogen peroxide solution, a sodium hypochlorite solution, a calcium hypochlorite solution, a sodium perborate solution, and combinations thereof. In an exemplary embodiment, the bleaching agent is a hydrogen peroxide solution having a concentration ranging from 2 wt % to 4 wt %. In another exemplary embodiment, the bleaching agent is a 2 wt % hydrogen peroxide solution.

In certain embodiments, in step (e), the softening treatment may be conducted using an anionic oil for 90 minutes. In an exemplary embodiment, the anionic oil is a phosphorylated synthetic fat-liquoring agent (Product name: phosphorylated synthetic oil; Trade name: SOFCON SWK) purchased from Giant Sun Fat & Oil Chemical Co., Ltd.

In certain embodiments, in step (e), the drying treatment may be conducted at a temperature ranging from 40° C. to 100° C. for a time period ranging from 90 minutes to 120 minutes. In an exemplary embodiment, the drying treatment is conducted at 100° C. for 120 minutes.

In certain embodiments, the fiber material obtained in step (e) may be further subjected to a bale opening treatment, so as to obtain two fibers having tow finenesses of about 0.9-5.0 decitex (dtex) and 40-60 dtex, respectively. In an exemplary embodiment, the fiber material contains 80 wt % of the fiber having a tow fineness of about 0.9-5.0 dtex and 20 wt % of the fiber having a tow fineness of about 40-60 dtex.

The present disclosure also provides a fiber material, which is prepared by a method described above.

According to the present disclosure, the fiber material may be further subjected to a processing treatment, so as to obtain a textile product. Examples of the textile product may include, but are not limited to, yarns, cotton yarns, and plaits (such as clothes, canvas, cord fabrics, nonwovens, and shoelaces).

According to the present disclosure, the fiber material may be manufactured into a fiber product using techniques well-known to those skilled in the paper industry or the artificial fiber industry. Examples of the fiber product may include, but are not limited to, papers, furniture, tableware, shoes, leathers, and filter media.

The disclosure will be further described by way of the following examples. However, it should be understood that the following examples are solely intended for the purpose of illustration and should not be construed as limiting the disclosure in practice.

### EXAMPLES

### General Experimental Materials:

### 1. Sources of Banana Pseudostem

The banana pseudostems used in the following experiments were collected from Nantou County, Jiji Township, Taiwan; Yunlin County, Citong Township, Taiwan; Kaohsiung City, Dashu District, Taiwan; and Pingtung County, Changzhi Township, Taiwan.

2. Saccharomyces cerevisiae Powder and Pectinase

Saccharomyces cerevisiae powder (Product name: Fermentis SafAle™ US-05 ale yeast; Manufacturer: Lesaffre Yeast Corporation) and pectinase (Product name: Pectic Enzyme; Manufacturer: North Mountain Supply) used in the following experiments were purchased from Bake n Brew Homebrew Supply.

### Example 1. Preparation of Fiber Material of the Present Disclosure

A. Preparation of Saccharomyces cerevisiae Inoculum

0.122 kg of Saccharomyces cerevisiae powder was evenly mixed with 6 kg of granulated sugar (purchased from Taiwan Sugar Corporation) and 18 L of reverse osmosis water, so as to obtain a Saccharomyces cerevisiae inoculum.

### B. Preparation of Fiber Material

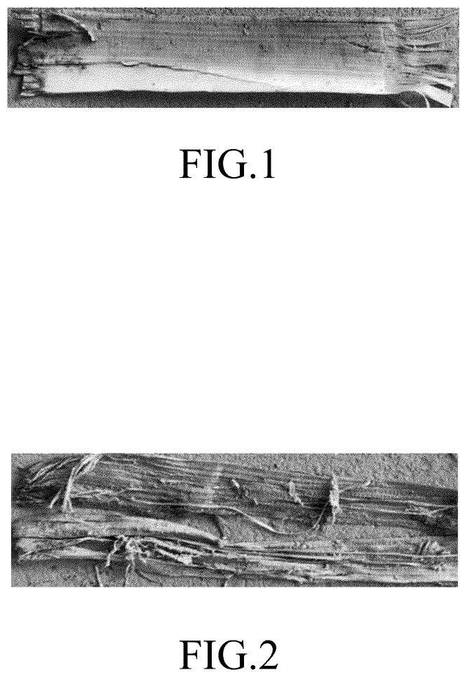

First, 30 kg of the banana pseudostems described in section 1 of “General Experimental Materials” were cut into segments, each having a length ranging from 60 cm to 100 cm (as shown in FIG. 1), by using a cutting machine. Next, the segmented banana pseudostems were subjected to a pressing treatment, so as to obtain pressed banana pseudostems (as shown in FIG. 2).

The pressed banana pseudostems were placed in a fermenter, and then the Saccharomyces cerevisiae inoculum prepared in section A of this example was inoculated in an amount of 0.5% (v/v) into the fermenter, followed by conducting a fermentation reaction at 28° C. for 120 hours, so as to obtain a fermented culture. During the fermentation reaction, the sugar content (degrees Brix) was measured using a saccharometer, and the sugar content was maintained at 18 degrees Brix to 20 degrees Brix by adding granulated sugar.

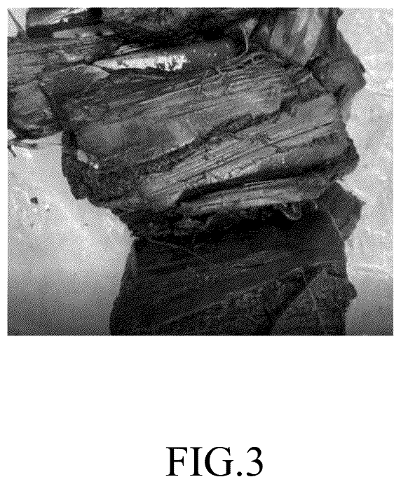

Thereafter, 0.027 mL of a 0.005 wt % pectinase solution was added to the fermented culture and mixed evenly, followed by conducting a simultaneous saccharification and fermentation process at 50° C. for 120 hours, so as to obtain a fermented product (as shown in FIG. 3). The fermented product was then subjected to a solid-liquid separation treatment using a filter for 2 hours, so as to obtain a solid fraction.

Afterward, the solid fraction was subjected to an alkali treatment by immersing the solid fraction in a 2 wt % potassium hydroxide solution at 90° C. for 3 hours, followed by conducting a water washing treatment with a high-pressure water column having a pressure of 100 bar, so as to remove lignin.

Next, the resultant washed solid substance was subjected to a bleaching treatment by immersing the washed solid substance in a 2 wt % hydrogen peroxide solution at 100° C. for 3 hours. The resultant bleached solid substance was subjected to a softening treatment by immersing the bleached solid substance in an appropriate amount of a phosphorylated synthetic fat-liquoring agent (Product name: phosphorylated synthetic oil; Trade name: SOFCON SWK; Manufacturer: Giant Sun Fat & Oil Chemical Co., Ltd) for 90 minutes, followed by conducting a drying treatment, which was performed at 100° C. for 120 minutes using a hot air circulator, so as to obtain a fiber tow.

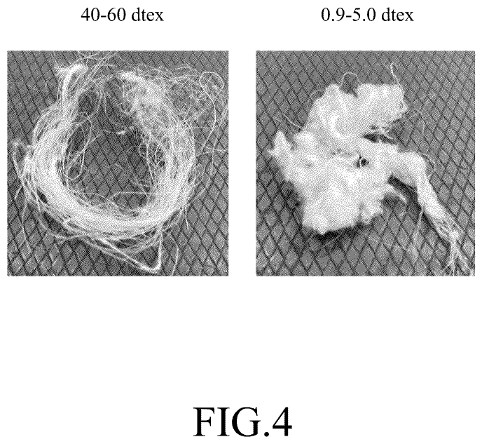

Thereafter, the fiber tow was subjected to a bale opening treatment, so as to obtain a fiber material having two different fibers with fiber fineness of about 0.9-5.0 decitex (dtex) and 40-60 dtex, respectively (as shown in FIG. 4). The fiber material served as an experimental group.

In addition, the fiber material serving as a comparative group was prepared for comparison purpose using procedure that is substantially similar to that of the experimental group, except that the banana pseudostems were not subjected to a pressing treatment, and the lignin and hemicellulose of the banana pseudostems were scraped using techniques well-known to those skilled in the art.

### C. Determination of Fiber Yield

The fiber material of each group prepared in section B of this example was subjected to weight measurement, and the fiber yield was calculated using the following Equation (I):

A=(B/C)×100 (I)
where A=fiber yield (%)
   B=total weight of fiber material of respective group
   C=total weight of banana pseudostems (i.e., 30 kg)

As shown in Table 1 below, the fiber yield determined in the experimental group was higher than that determined in the comparative group. In addition, the applicant found that the fiber material of the comparative group did not have fibers with fiber fineness of lower than 4 dtex.

These results indicate that the fiber material prepared according to the method of the present disclosure has more fiber content and fiber types, and hence can be effectively recycled.

TABLE 1

| Group | Fiber yield (%) |
|---|---|
| Experimental group | 8 |
| Comparative group | 2 |

### Example 2. Recycling of Fermented Culture

### A. Preparation of Bio-Alcohol and Distillation Residue

The fermented culture obtained in section B of Example 1 was subjected to filtration using a filter to obtain a filtrate. The filtrate was then subjected to distillation at 78° C. to 100° C. using a distillation machine, so as to obtain a bio-alcohol and a distillation residue.

### B. Analysis of Amino Acid Components

The distillation residue obtained in section A of this example was subjected to amino acid component analysis which was entrusted to Jackson International Standard Inspection Co., Ltd., Taiwan.

As shown in Table 2 below, the distillation residue contained various amino acids.

TABLE 2

| Type of amino acid | Content (mg/100 g) |
|---|---|
| Alanine | 156.02 |
| Arginine | 55.68 |
| Asparagine | 651 |
| Cystine | 480.1 |
| Glutamic acid | 247.38 |
| Glycine | 81.78 |
| Histidine | 20.15 |
| Isoleucine | 70.06 |
| Leucine | 105.01 |
| Lysine | 144.53 |
| Methionine | 8.03 |
| Phenylalanine | 60.08 |
| Proline | 55.06 |
| Threonine | 71.11 |
| Tyrosine | 41.58 |
| Valine | 87.1 |

### C. Analysis of Metal Components

The distillation residue obtained in section A of this example was subjected to metal component analysis which was entrusted to Jackson International Standard Inspection Co., Ltd., Taiwan.

As shown in Table 3 below, the metals and the contents thereof in the distillation residue were in compliance with the regulations for organic fertilizers in the Agriculture Fertilizer Standards stipulated by the Agriculture and Food Agency, Council of Agriculture, Executive Yuan, Taiwan.

TABLE 3

| Type of metal | Content | |
|---|---|---|
| Arsenic | 0.12 | ppm |
| Cadmium | 0 | ppm |
| Copper | 0.67 | ppm |
| Lead | 0.06 | ppm |
| Mercury | 0 | ppm |
| Chromium | 0.06 | ppm |
| Nickel | 0.14 | ppm |
| Sodium | 191 | ppm |
| Zinc | 12 | ppm |

| | |
|---|---|
| Potassium peroxide | 0.81% |
| Sodium chloride | 0.035% |

Summarizing the above test results, it is clear that the method of the present disclosure can effectively prepare a fiber material from a banana pseudostem, and the fermented culture obtained during the preparation process can be further processed by distillation to obtain a bio-alcohol and a distillation residue that can be used as an organic fertilizer.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for preparing a fiber material from a banana pseudostem, comprising:
(a) subjecting the banana pseudostem to a pressing treatment, so as to obtain a pressed banana pseudostem;
(b) subjecting the pressed banana pseudostem to a fermentation reaction with Saccharomyces cerevisiae, so as to obtain a fermented culture;
(c) subjecting the fermented culture to a simultaneous saccharification and fermentation process with pectinase, so as to obtain a fermented product;
(d) subjecting the fermented product to a solid-liquid separation treatment, so as to obtain a solid fraction; and
(e) subjecting the solid fraction to an alkali treatment, a water washing treatment, a bleaching treatment, a softening treatment, and a drying treatment in sequence, so as to obtain the fiber material.

2. The method as claimed in claim 1, wherein in step (b), the fermentation reaction is conducted at a temperature ranging from 20° C. to 32° C.

3. The method as claimed in claim 1, wherein in step (c), the simultaneous saccharification and fermentation process is conducted at a temperature ranging from 15° C. to 55° C.

4. The method as claimed in claim 1, further comprising: subjecting the fiber material obtained in step (e) to a bale opening treatment.

5. A fiber material, which is prepared by a method according to claim 1.
